(19) United States
(12) Patent Application Publication
(10) Pub. No.: US 2025/0352586 A1
(43) Pub. Date: Nov. 20, 2025

(54) COMPOSITIONS FOR TREATING AND/OR RELIEVING SARCOPENIA AND USES THEREOF

(71) Applicant: SPACE ARK BIOMEDICAL CO., LTD., Taipei City (TW)

(72) Inventors: JUI-JEN CHANG, Taipei City (TW); CHIEH-CHEN HUANG, Taichung City (TW); EN-PEI CHIANG, Taichung City (TW)

(73) Assignee: SPACE ARK BIOMEDICAL CO., LTD., Taipei City (TW)

(21) Appl. No.: 19/196,311

(22) Filed: May 1, 2025

(30) Foreign Application Priority Data
May 17, 2024 (TW) 113118459
Mar. 4, 2025 (TW) 114107856

(51) Int. Cl. A61K 35/742

(52) U.S. Cl. CPC A61K 35/742

(57) ABSTRACT

A composition for treating and/or relieving sarcopenia includes Clostridium butyricum M1. A microbial number of Clostridium butyricum M1 ranges from 10⁶ CFU/g to 10¹² CFU/g. The composition further includes Bacillus coagulans CC212. Bacillus coagulans CC212 is deposited in Bioresource Collection and Research Center. A deposit number of Bacillus coagulans CC212 is BCRC911233. A microbial number of Bacillus coagulans CC212 is ranges from 10⁶ CFU/g to 10¹² CFU/g. A use of the composition is to orally administer the composition to a subject. Clostridium butyricum M1 release a Clostridium butyricum exosome. The Clostridium butyricum exosome could promote muscle growth of the subject. Bacillus coagulans CC212 release a Bacillus coagulans exosome. The Bacillus coagulans exosome and the Clostridium butyricum exosome could effectively reduce a fat mass of the subject. Therefore, the subject could experience an increase in muscle mass and a reduction in fat after taking the composition.

### BACKGROUND OF THE INVENTION

### Technical Field

The present invention relates generally to probiotic compositions, and more particularly to compositions for treating and/or relieving sarcopenia and uses thereof.

### Description of Related Art

With respect to health issues in an aging society, sarcopenia is considered one of the main causes of deteriorating health in the elderly. Sarcopenia is a syndrome including progressive and generalized loss of skeletal muscle mass and functions. The causes of sarcopenia include the degeneration of motor neurons, nutritional imbalance, a decrease in protein synthesis, chronic diseases and/or inflammation responses, etc. Patients suffering from sarcopenia experience reduced mobility, a lower quality of life and an increased risk of disability and falls.

However, there is no medication for treating sarcopenia in clinical practice. It is known that exercising and taking supplementary protein and calories could relieve sarcopenia. For elderly patients, intake of nutrient could not be effectively and completely absorbed, such that muscle mass of the elderly patients could not be effectively increased. Therefore, how to provide food compositions to effectively relieve sarcopenia, is a problem needed to be solved in the industry.

### BRIEF SUMMARY OF THE INVENTION

In view of the above, the primary objective of the present invention is to provide a composition for treating and/or relieving sarcopenia and a method of treating and/or relieving sarcopenia by using a composition for treating and/or relieving sarcopenia, wherein the composition could effectively promote muscle growth of a subject and reduce fat accumulation, thereby relieving sarcopenia of the subject.

The present invention provides a composition for treating and/or relieving sarcopenia, including Clostridium butyricum M1, wherein a microbial number of Clostridium butyricum M1 ranges from 10⁶ CFU/g to 10¹² CFU/g.

In an embodiment, the composition includes Bacillus coagulans CC212; Bacillus coagulans CC212 is deposited Bioresource Collection and Research Center; a deposit number of Bacillus coagulans CC212 is BCRC911233; a microbial number of Bacillus coagulans CC212 ranges from 10⁶ CFU/g to 10¹² CFU/g.

In an embodiment, the composition further includes Bacillus coagulans endospores and Clostridium butyricum endospores; the Bacillus coagulans endospores are formed by Bacillus coagulans CC212; the Clostridium butyricum endospores are formed by Clostridium butyricum M1; a ratio of a combined weight of Bacillus coagulans CC212 and the Bacillus coagulans endospores to a combined weight of Clostridium butyricum M1 and the Clostridium butyricum endospores is between 1:0.05 and 1:10.

The present invention further provides a method of treating and/or relieving sarcopenia by using a composition for treating and/or relieving sarcopenia. The method includes orally administering the composition to a subject. The composition includes Clostridium butyricum M1. A microbial number of Clostridium butyricum M1 ranges from 10⁶ CFU/g to 10¹² CFU/g. Clostridium butyricum M1 release a Clostridium butyricum exosome. The Clostridium butyricum exosome includes butyric acid to activate vitamin B6 of the subject, thereby increasing a muscle mass of the subject.

In an embodiment, the composition includes Clostridium butyricum endospores; the Clostridium butyricum endospores are formed by Clostridium butyricum M1; when the composition is administered to the subject, the Clostridium butyricum endospores undergo germination and release the Clostridium butyricum exosome.

In an embodiment, the composition includes Bacillus coagulans CC212; a microbial number of Bacillus coagulans CC212 ranges from 10⁶ CFU/g to 10¹² CFU/g; Bacillus coagulans CC212 is deposited in Bioresource Collection and Research Center; a deposit number of Bacillus coagulans CC212 is BCRC911233; when the composition is administered to the subject, Bacillus coagulans CC212 releases a Bacillus coagulans exosome; the Bacillus coagulans exosome and the Clostridium butyricum exosome are absorbed by the subject, thereby decreasing a fat mass of the subject.

With the aforementioned design, Clostridium butyricum M1 of the composition could release the Clostridium butyricum exosome in the subject. The Clostridium butyricum exosome is absorbed by the subject to activate vitamin B6 in the subject, thereby promoting muscle growth of the subject. The composition includes the combination of Bacillus coagulans CC212 and Clostridium butyricum M1. The Bacillus coagulans exosome and the Clostridium butyricum exosome are absorbed by the subject to reduce fat accumulation. In this way, after taking the composition, the subject could experience an increase in muscle mass and a reduction in fat, thereby treating or relieving sarcopenia.

Moreover, the composition includes the addition of the Bacillus coagulans endospores and the Clostridium butyricum endospores. As the Bacillus coagulans endospores and the Clostridium butyricum endospores has high resistances to high temperatures and acid and alkali, so that the Bacillus coagulans endospores and the Clostridium butyricum endospores could pass through the stomach of the subject. The Bacillus coagulans endospores and the Clostridium butyricum endospores could undergo germination in the intestines of the subject to reproduce Bacillus coagulans CC212 and Clostridium butyricum M1, so that the growth of Bacillus coagulans CC212 and Clostridium butyricum M1 is promoted in the intestines of the subject, thereby promoting muscle growth of the subject and reducing fat accumulation.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1 is a bar chart showing the perirenal fat weights of the subjects of the normal control group, the vehicle group, and the experimental groups 1 and 2 according to an embodiment of the present invention;

FIG. 2 is a bar chart showing the periovarian fat weights of the subjects of the normal control group, the vehicle group, and the experimental groups 1 and 2 according to the embodiment of the present invention;

FIG. 3A is a schematic view showing the particle size distribution of the Clostridium butyricum exosome sample according to the embodiment of the present invention;

FIG. 3B is a schematic view showing the staining distribution of the Clostridium butyricum exosome sample according to the embodiment of the present invention;

FIG. 4A is a bar chart showing the spore viabilities of the Clostridium butyricum endospores of the control group and the experimental group in the acid resistance experiment according to the embodiment of the present invention; and

FIG. 4B is a bar chart showing the spore viability of the Clostridium butyricum endospores of the control group and the experimental group in the bile salt resistance experiment according to the embodiment of the present invention.

### DETAILED DESCRIPTION OF THE INVENTION

A composition for treating and/or relieving sarcopenia according to an embodiment of the present invention includes Clostridium butyricum M1. A deposit number of Clostridium butyricum M1 is BCRC910362. Clostridium butyricum M1 is selected and obtained by cultivating brewer's spent grain. A microbial number of Clostridium butyricum M1 ranges from 10⁶ CFU/g to 10¹² CFU/g. Preferably, the microbial number of Clostridium butyricum M1 ranges from 10⁷ CFU/g to 10¹² CFU/g. Clostridium butyricum M1 is Clostridium clostridioforme that has a high acid resistance and favors anaerobic conditions. Clostridium butyricum M1 could live normally in intestines of animals. In an embodiment, the composition further includes Clostridium butyricum endospores. The Clostridium butyricum endospores are obtained by cultivating Clostridium butyricum M1 in a fermenter. A number of spores of the Clostridium butyricum endospores is greater than 10⁷/g. A content of the Clostridium butyricum endospores accounts for between 30 wt % and 90 wt % of a total content of the composition. A content of Clostridium butyricum M1 accounts for between 10 wt % and 70 wt % of the total content of the composition. In extreme conditions, Clostridium butyricum M1 undergoes sporulation to form the Clostridium butyricum endospores. The Clostridium butyricum endospores could survive in extreme conditions, thereby lengthening the viability of the Clostridium butyricum endospores in the composition. When the Clostridium butyricum endospores are situated in a condition favorable for growth, the Clostridium butyricum endospores undergo germination to reproduce Clostridium butyricum M1.

Many researches show that Clostridium butyricum M1 mainly form a butyrate metabolite in the intestines of the animals. The butyrate metabolite could promote the growth of intestinal mucosal cells in the animals and reduce the formation of fat. Moreover, the butyrate metabolite could activate the mTOR mechanism in bodies of the animals to inhibit the proteolysis of ubiquitin proteasome, so that muscle hypertrophy could be promoted and apoptosis of muscle cells at catabolic state and muscular atrophy could be reduced and slowed, thereby increasing the muscle mass of the animals.

Additionally, the butyrate metabolite formed by Clostridium butyricum M1 could reduce the secretion of inflammatory cytokines, such as IL-6 and TNF-α, of macrophages in fat tissues, thereby preventing or relieving muscle loss.

In an embodiment, the composition further includes Bacillus coagulans CC212. Bacillus coagulans CC212 is deposited in Bioresource Collection and Research Center (BCRC). A deposit number of Bacillus coagulans CC212 is BCRC911233. Bacillus coagulans CC212 is similarly selected and obtained by cultivating brewer's spent grain. A microbial number of Bacillus coagulans CC212 ranges from 10⁶ CFU/g to 10¹² CFU/g. Preferably, the microbial number of Bacillus coagulans CC212 ranges from 10⁷ CFU/g to 10¹² CFU/g. Researches show that Bacillus coagulans CC212 is aerobic lactic acid bacteria. Bacillus coagulans CC212 could combine with cholesterol in the intestines in the animal and inhibit the protease required for forming cholesterol, thereby facilitating the reduction of blood cholesterol level and decreasing fat absorption and storage.

In an embodiment, the composition further includes Bacillus coagulans endospores. The Bacillus coagulans endospores are formed by cultivating Bacillus coagulans CC212 in a fermenter. A number of spores of the Bacillus coagulans endospores is greater than 10⁷/g. The Bacillus coagulans endospores could survive in extreme conditions. When the Bacillus coagulans endospores are situated in a condition favorable for growth, the Bacillus coagulans endospores could undergo germination to reproduce Bacillus coagulans CC212.

In the current embodiment, when the composition includes Clostridium butyricum M1 and Bacillus coagulans CC212 but does not include the Clostridium butyricum endospores and the Bacillus coagulans endospores, a content of Clostridium butyricum M1 accounts for between 5 wt % and 95 wt % of the total content of the composition, and a content of Bacillus coagulans CC212 accounts for between 5 wt % and 95 wt % of the total content of the composition, wherein a weight ratio of Bacillus coagulans CC212 to Clostridium butyricum M1 is between 1:0.05 and 1:10. In an embodiment, the weight ratio of Bacillus coagulans CC212 to Clostridium butyricum M1 is between 1:0.1 and 1:1, i.e., the content of Clostridium butyricum M1 accounts for between 10 wt % and 50 wt % of the total content of the composition and the content of Bacillus coagulans CC212 accounts for between 50 wt % and 90 wt % of the total content of the composition.

In another embodiment, when the composition includes Bacillus coagulans CC212, the Bacillus coagulans endospores, Clostridium butyricum M1, and the Clostridium butyricum endospores, a ratio of a combined weight of Bacillus coagulans CC212 and the Bacillus coagulans endospores to a combined weight of Clostridium butyricum M1 and the Clostridium butyricum endospores is between 1:0.05 and 1:10. The content of the Clostridium butyricum endospores accounts for between 30 wt % and 90 wt % of a combined content of Clostridium butyricum M1 and the Clostridium butyricum endospores. The content of Clostridium butyricum M1 accounts for between 10 wt % and 70 wt % of the combined content of Clostridium butyricum M1 and the Clostridium butyricum endospores. The content of the Bacillus coagulans endospores accounts for between 50 wt % and 90 wt % of a combined content of Bacillus coagulans CC212 and the Bacillus coagulans endospores. The content of Bacillus coagulans CC212 accounts for between 10 wt % and 50 wt % of the combined content of Bacillus coagulans CC212 and the Bacillus coagulans endospores.

Moreover, the present invention further provides a method for treating and/or relieving sarcopenia by using a composition for treating and/or relieving sarcopenia. The method includes orally administering the aforementioned composition to a subject. Clostridium butyricum M1 in the composition releases a Clostridium butyricum exosome in the subject. The Clostridium butyricum exosome includes butyric acid to activate vitamin B6 of the subject, thereby increasing a muscle mass of the subject. When the composition includes Bacillus coagulans CC212, Bacillus coagulans CC212 and Clostridium butyricum M1 are activated, and Bacillus coagulans CC212 releases a Bacillus coagulans exosome. The Bacillus coagulans exosome and the Clostridium butyricum exosome are absorbed by the subject, thereby decreasing a fat mass of the subject. In still another embodiment, when the composition further includes the Bacillus coagulans endospores and the Clostridium butyricum endospores, the Bacillus coagulans endospores and the Clostridium butyricum endospores could respectively undergo germination in the intestines of the subject and release the Bacillus coagulans exosome and the Clostridium butyricum exosome.

The term “subject” in the specification refers to an animal including the human species that is treatable with the composition of the current embodiment. The term “subject”, unless otherwise stated in the specification, is intended to refer to an animal that could produce therapeutic benefits after being treated with the composition of the current embodiment, such as human, rat, mouse, guinea pig, monkey, pig, goat, cow, horse, dog, cat, chicken, bird, and fowl. In the current embodiment, the “subject” refers to chicken and mouse for experiments.

The composition is orally administered by adding the composition to a food of the subject. Based on a weight of the food of the subject, an amount of the composition added ranges from 0.05 wt % to 1.0 wt %. In an embodiment, the amount of the composition added ranges from 0.1 wt % to 0.5 wt % of the weight of the food of the subject. The composition could at least include Clostridium butyricum M1. In the current embodiment, the composition includes Clostridium butyricum M1 and Bacillus coagulans CC212; the microbial number of Clostridium butyricum M1 and the microbial number of Bacillus coagulans CC212 could be adjusted based on a weight of the subject. In another embodiment, the composition includes Bacillus coagulans CC212, the Bacillus coagulans endospores, Clostridium butyricum M1, and the Clostridium butyricum endospores; the number of spores of the Clostridium butyricum endospores and the number of spores of the Bacillus coagulans endospores could be adjusted based on the weight of the subject.

In this way, Clostridium butyricum M1 of the composition could release the Clostridium butyricum exosome in the subject. The Clostridium butyricum exosome is absorbed by the subject to activate vitamin B6 of the subject, thereby facilitating muscle growth of the subject. When the composition includes Bacillus coagulans CC212 and Clostridium butyricum M1, the Bacillus coagulans exosome and the Clostridium butyricum exosome are absorbed by the subject to reduce fat accumulation, so that the subject could experience an increase of muscle mass and a reduction of fat after taking the composition, thereby treating or relieving sarcopenia.

Moreover, the composition includes the Bacillus coagulans endospores and the Clostridium butyricum endospores. As the Bacillus coagulans endospores and the Clostridium butyricum endospores could withstand high temperatures and acidic and alkaline conditions, the Bacillus coagulans endospores and the Clostridium butyricum endospore could pass through the stomach environment of the subject and could undergo germination in the intestines of the subject to reproduce Bacillus coagulans CC212 and Clostridium butyricum M1, so that the growth of Bacillus coagulans CC212 Clostridium butyricum M1 in the intestines of the subject is promoted, thereby promoting muscle growth of the subject and reducing fat accumulation.

In order to thoroughly demonstrate the primary objective, features, and effects of the present invention, the composition of the current embodiment is subjected to animal experiments, thereby illustrating that the composition could promote muscle growth and reduce fat accumulation.

1. Measuring changes in muscle mass and fat mass after the subject is fed with the composition:

The “subject” in the current experiment is chickens. The chickens are black feather native chickens purchased from 18 Ranch. The composition is added to the food of the subject as a feed.

The current experiment includes a normal control group and experimental groups 1 to 4:

The normal control group: the subject (chicken) is basically fed with a feed three times a day, with 75 g of feed per feeding.

The experimental group 1: the composition is added to the feed; the composition is powders of Clostridium butyricum M1; based on a weight of the food (feed) of the subject, the amount of the composition added is 0.1 wt %; the subject (chicken) is fed three times a day, with 75 g of feed, which includes the composition, per feeding.

The experimental group 2: the composition is added to the feed; the composition is powders of Clostridium butyricum M1; based on a weight of the food (feed) of the subject, the amount of the composition added is 0.5 wt %; the subject (chicken) is fed three times a day, with 75 g of feed, which includes the composition, per feeding.

The experimental group 3: the composition is added to the feed; the composition is combined powders of Clostridium butyricum M1 and Bacillus coagulans CC212; the weight ratio of Clostridium butyricum M1 to Bacillus coagulans CC212 is 1:1; based on a weight of the food (feed), the amount of the composition added is 0.1 wt %; the subject (chicken) is fed three times a day, with 75 g of feed, which includes the composition, per feeding.

The experimental group 4: the composition is added to the feed; the composition is combined powders of Clostridium butyricum M1 and Bacillus coagulans CC212; the weight ratio of Clostridium butyricum M1 to Bacillus coagulans CC212 is 1:1; based on a weight of the food (feed), the amount of the composition added is 0.5 wt %; the subject (chicken) is fed three times a day, with 75 g of feed, which includes the composition, per feeding.

In the current experiment, Bacillus coagulans CC212 and Clostridium butyricum M1 are jointly inoculated in a MRS medium for liquid fermentation. The cultivation conditions include a fermentation temperature of 37° C., 6.5 pH value, and 48-hour anaerobic cultivation. Afterwards, the MRS medium is separated to obtain strains of Bacillus coagulans CC212 and strains of Clostridium butyricum M1. Freeze drying is performed on the strains of Bacillus coagulans CC212 and the strains of Clostridium butyricum M1 respectively, thereby obtaining the powders of Bacillus coagulans CC212 and the powders of Clostridium butyricum M1. The microbial number of Bacillus coagulans CC212 is greater than 109 CFU/g. The microbial number of Clostridium butyricum M1 is greater than 109 CFU/g. The composition in the current experiment does not include the addition of the Bacillus coagulans endospores and the Clostridium butyricum endospores.

Models for the muscle experiment and the fat experiment are to continuously feed the subjects (chickens) of the normal control group and the experimental groups 1 to 4 for 3 months and sacrifice the subjects (chickens) of the normal control group and the experimental groups 1 to 4 for measuring a chicken breast weight, an abdominal fat weight, a subcutaneous fat weight, and a total body fat weight of the subjects (chickens).

Table 1 shows data of the chicken breast weights of the subjects (chickens) of the normal control group and the experimental groups 1 to 4. When the subjects (chickens) of the normal control group and the experimental groups 1 to 4 have been fed for 3 months, the chicken breast weight of the experimental group 1 is greatly increased by 20% compared to the chicken breast weight of the normal control group; the chicken breast weight of the experimental group 2 is greatly increased by 3% compared to the chicken breast weight of the normal control; the chicken breast weight of the experimental group 3 is greatly increased by 28% compared to the chicken breast weight of the normal control group; the chicken breast weight of the experimental group 4 is greatly increased by 14% compared to the chicken breast weight of the normal control group. It could be seen form the measurement results that Clostridium butyricum M1 in the compositions used in the experimental groups 1 and 2 could increase the muscle mass of the subject (chicken). The combination of Clostridium butyricum M1 and Bacillus coagulans CC212 in the compositions used in the experimental groups 3 and 4 could further increase the muscle mass of the subject (chicken).

TABLE 1
data table of the chicken breast weights of the subjects of
the normal control group and the experimental groups 1 to 4

Time
Feeding for 3 months

| | Chicken breast | Percentage change in the |
|---|---|---|
| Item | weight ratio (%) | chicken breast weight |
| Normal control | 7.4 ± 0.43 | — |
| group | | |
| Experimental | 8.88 ± 0.96 | +20% |
| group 1 | | |
| Experimental | 7.58 ± 0.94 | +3% |
| group 2 | | |
| Experimental | 9.43 ± 1.73 | +28% |
| group 3 | | |
| Experimental | 8.41 ± 1.63 | +14% |
| group 4 | | |

Moreover, Table 2 shows data of the abdominal fat weights of the normal control group and the experimental groups 1 to 4. When the subjects (chickens) of the normal control group and the experimental groups 1 to 4 have been fed for 3 months, the abdominal fat weight of the experimental group 1 is greatly increased compared to the abdominal fat weight of the normal control group; the abdominal fat weight of the experimental group 2 is slightly decreased compared to the abdominal fat weight of the normal control group; the abdominal fat weights of the experimental groups 3 and 4 are greatly decreased compared to the abdominal fat weight of the normal control group. It could be seen from the measurement results that the effect of Clostridium butyricum M1 of the composition in reducing abdominal fat of the subject (chicken) is limited. When the composition including the combination of Clostridium butyricum M1 and Bacillus coagulans CC212 is administered to the subject (chicken), the abdominal fat weight of the subject (chicken) could be greatly reduced.

TABLE 2
data table of the abdominal fat weights of the subjects of
the normal control group and the experimental groups 1 to 4

| | Time |
|---|---|
| | Feeding for 3 months |
| Item | Abdominal fat weight ratio (%) |
| Normal control group | 3.96 ± 1.71 |
| Experimental group 1 | 4.19 ± 0.78 |
| Experimental group 2 | 3.25 ± 3.27 |
| Experimental group 3 | 2.22 ± 1.80 |
| Experimental group 4 | 2.08 ± 0.87 |

Table 3 shows data of the subcutaneous fat weights of the subjects (chickens) of the normal control group and the experimental groups 1 to 4. When the subjects (chickens) of the normal control group and the experimental groups 1 to 4 have been fed for 3 months, the subcutaneous fat weights of the experimental groups 1 and 2 are slightly increased compared to the subcutaneous fat weight of the normal control group; the subcutaneous fat weights of the experimental groups 3 and 4 are greatly decreased compared to the subcutaneous fat weight of the normal control group. It could be seen from the measurement results that the effect of Clostridium butyricum M1 of the composition in reducing subcutaneous fat of the subject (chicken) is limited. When the composition including the combination of Clostridium butyricum M1 and Bacillus coagulans CC212 is administered to the subject (chicken), the subcutaneous fat weight of the subject (chicken) could be greatly reduced.

TABLE 3
data table of the subcutaneous fat weights of the subjects of
the normal control group and the experimental groups 1 to 4

| | Time |
|---|---|
| | Feeding for 3 months |
| Item | Subcutaneous fat weight ratio (%) |
| Normal control group | 1.04 ± 0.41 |
| Experimental group 1 | 1.23 ± 0.49 |
| Experimental group 2 | 1.17 ± 1.18 |
| Experimental group 3 | 0.73 ± 0.64 |
| Experimental group 4 | 0.99 ± 0.45 |

Table 4 shows data of the total body fat weights of the subjects (chickens) of the normal control group of the experimental groups 1 to 4. When the subjects (chickens) of the normal control group and the experimental groups 1 to 4 have been fed for 3 months, the total body fat weight of the experimental group 1 is slightly increased compared to the total body fat weight of the normal control group; the total body fat weight of the experimental group 2 is slightly decreased compared to the total body fat weight of the normal control group; the total body fat weights of the experimental groups 3 and 4 are greatly decreased compared to the total body fat weight of the normal control group. It could be seen from the measurement results that when the composition including the combination of Clostridium butyricum M1 and Bacillus coagulans CC212 is administered to the subject (chicken), the total body fat weight of the subject (chicken) could be greatly reduced, thereby reducing fat accumulation.

TABLE 4
data table of the total body fat weights of the normal
control group and the experimental groups 1 to 4

| | Time |
|---|---|
| | Feeding for 3 months |
| Item | Total body fat weight ratio (%) |
| Normal control group | 5.00 ± 2.02 |
| Experimental group 1 | 5.42 ± 0.98 |
| Experimental group 2 | 4.42 ± 4.46 |
| Experimental group 3 | 2.95 ± 2.43 |
| Experimental group 4 | 3.07 ± 1.32 |

In summery, Clostridium butyricum M1 in the composition could greatly promote the muscle growth of the subject (chicken). When the composition including the combination of Clostridium butyricum M1 and Bacillus coagulans CC212 is administered to the subject (chicken), the muscle growth of the subject (chicken) could be further promoted. Moreover, the combination of Clostridium butyricum M1 and Bacillus coagulans CC212 could greatly reduce the fat mass of the subject (chicken) and reduce fat accumulation, so that the subject could experience an increase of muscle mass and a reduction of fat after taking the composition, thereby treating or relieving sarcopenia.

2. Measuring changes in perirenal fats and periovarian fats after the subject is fed with the composition:

The “subject” in the current experiment is C57BL/6J mice aged between 8 weeks and 14 weeks, purchased from the National Laboratory Animal Center.

The current experiment includes a normal control group, a vehicle group, and experimental groups 1 and 2.

The normal control group: the subject (mouse) is fed with regular feed three times a day at fixed times and in fixed amounts.

The vehicle group: Leptin at 0.15 mg/kg is added to the feed; the subject (mouse) is fed with the feed, which includes Leptin, three times a day at fixed times and in fixed amounts.

The experimental group 1: the composition is added to the feed; the composition is powders of Clostridium butyricum M1; based on a weight of the food (feed) of the subject, the amount of the composition added is 0.1 wt %; the subject (mouse) is fed with the feed, which includes the composition, three times a day at fixed times and in fixed amounts

The experimental group 2: the composition is added to the feed; the composition is combined powders of Clostridium butyricum M1 and Bacillus coagulans CC212; the weight ratio of Clostridium butyricum M1 to Bacillus coagulans CC212 is 1:1; based on a weight of the food (feed), the amount of the composition added is 0.1 wt %; the subject (mouse) is fed with the feed, which includes the composition, three times a day at fixed times and in fixed amounts.

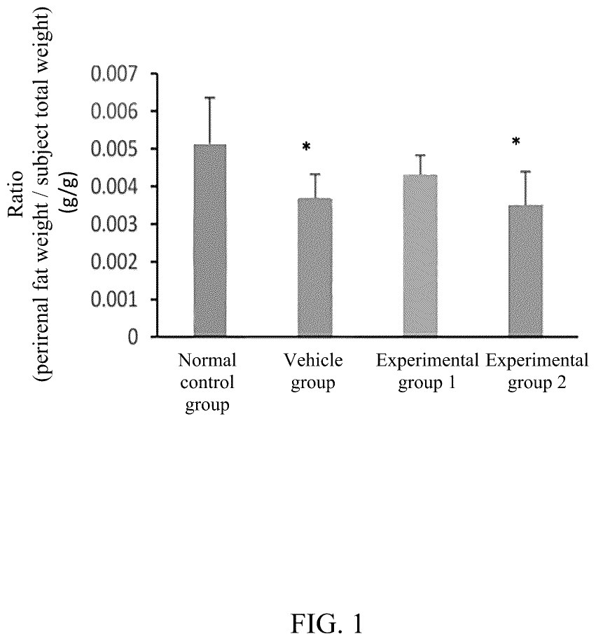

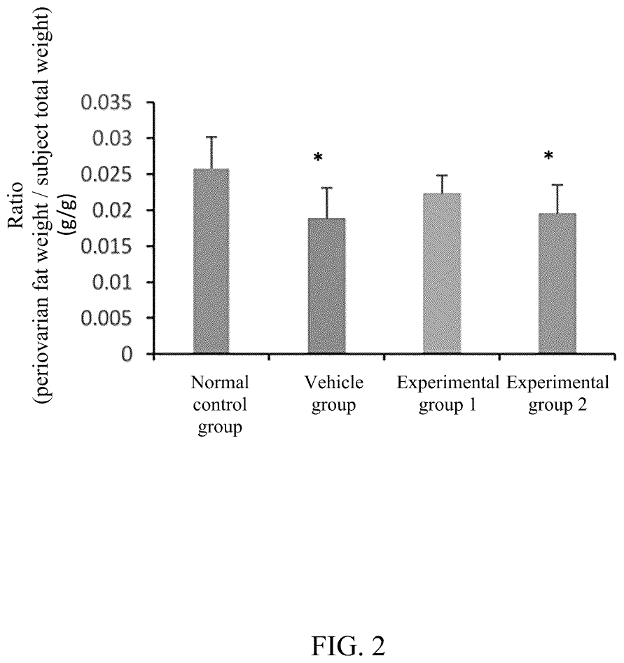

The normal control group, the vehicle group, and the experimental groups 1 and 2 have been fed for 60 days and are sacrificed for measuring the changes in the perirenal fats and the periovarian fats. FIG. 1 shows the perirenal fat weights of the subjects (mice) of the normal control group, the vehicle group, and the experimental groups 1 and 2. FIG. 2 shows the periovarian fat weights of the subjects (mice) of the normal control group, the vehicle group, and the experimental groups 1 and 2. The results of the data and standard errors (SE) shown in FIG. 1 and FIG. 2 are measurement results obtained by carrying out three independent experiments (n=3) on the normal control group, the vehicle group, and the experimental groups 1 and 2 respectively. The symbol “*” refers to a comparison between a value of the normal control group and a value of the vehicle group, or a comparison between a value of the normal control group and a value of the experimental groups 1 and 2. p<0.05.

It could be seen from FIG. 1 and FIG. 2 that the perirenal fat weight and the periovarian fat weight of the vehicle group are greatly reduced compared to the perirenal fat weight and the periovarian fat weight of the normal control group; the perirenal fat weight and the periovarian fat weight of the experimental group 1 are slightly decreased compared to the perirenal fat weight and the periovarian fat weight of the normal control group; the perirenal fat weight and the periovarian fat weight of the experimental group 2 are greatly decreased compared to the perirenal fat weight and the periovarian fat weight of the normal control group; the perirenal fat weight and the periovarian fat weight of the experimental group 2 are similar to the perirenal fat weight and the periovarian fat weight of the vehicle group, indicating that when the composition of the experimental group 2 including the combination of Clostridium butyricum M1 and Bacillus coagulans CC212 is administered to the subject (mouse), the perirenal fat weight and the periovarian fat weight of the subject (mouse) could be greatly reduced, thereby reducing visceral fat accumulation.

3. Analyzing the properties of Clostridium butyricum M1 in releasing the Clostridium butyricum exosome:

The current experiment determines whether Clostridium butyricum M1 releases the Clostridium butyricum exosome. Exosomes are vesicles with bilayer membrane structures having a particle size ranging from 30 nm to 200 nm and are formed by viable cells through exocytosis. The current experiment is to firstly cultivate Clostridium butyricum M1; a separation and sieving process is performed on a fermentation medium having Clostridium butyricum M1 to obtain a Clostridium butyricum exosome sample; particle centration, particle size distribution, and surface staining analyses are performed on the Clostridium butyricum exosome sample by using a flow nanoanalyzer (NanoFCM U30), wherein a standardized exosome sample (S16M-Exo) used by the flow nanoanalyzer has a particle size ranging from 68 nm to 155 nm.

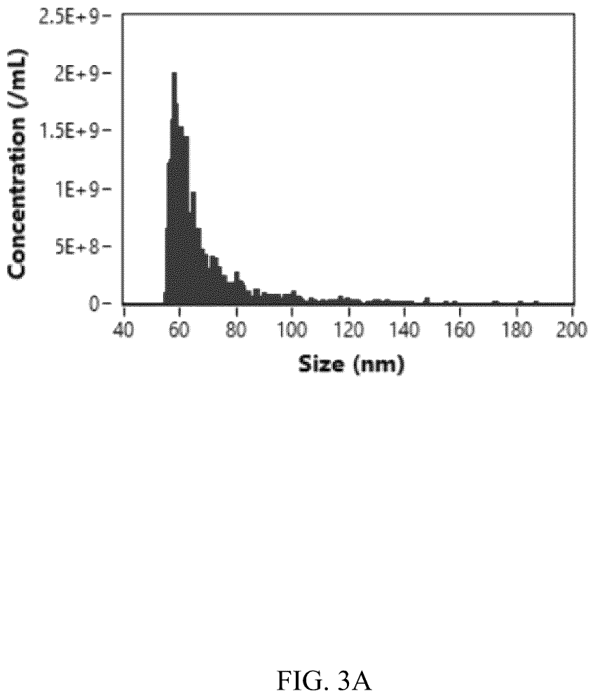

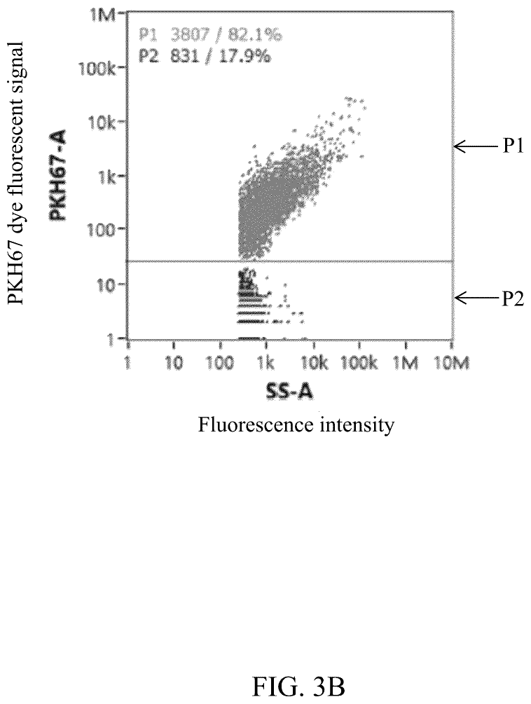

FIG. 3A shows a particle size distribution of the Clostridium butyricum exosome sample. The flow nanoanalyzer relatively measures bioparticles, which have a particle size ranging from 40 nm to 200 nm, in the Clostridium butyricum exosome sample based on a particle size condition of the standardized exosome sample. The bioparticles have a median particle size of 62.2 nm and an average particle size of 67.1 nm. A concentration of the bioparticles with the particle size ranging from 40 nm to 200 nm in the Clostridium butyricum exosome sample calculated by the nanoanalyzer is 3.77×10¹⁸ (particles/ml).

Marking and staining are performed on the Clostridium butyricum exosome sample at room temperature by using PKH67 dye. The PKH67 dye is mainly used to stain and track exosomes. Then, the stained Clostridium butyricum exosome sample is centrifuged and rinsed, followed by analysis. FIG. 3B shows a staining distribution of the Clostridium butyricum exosome sample. P1 is a number and a percentage of the bioparticles having PKH67 staining signals (green signal distribution) in the Clostridium butyricum exosome sample. P2 is a number and a percentage of the bioparticles without PKH67 staining signals (blue signal distribution) in the Clostridium butyricum exosome sample. It could be seen from the measurement results in FIG. 3B that 82.1% of the bioparticles in the Clostridium butyricum exosome sample have the bilayer membrane structures and 17.9% of the bioparticles in the Clostridium butyricum exosome sample does not have the bilayer membrane structures, proving that the Clostridium butyricum exosome sample could be provided with the Clostridium butyricum exosome by cultivating Clostridium butyricum M1.

The current experiment mainly illustrates that the Clostridium butyricum exosome could be obtained from Clostridium butyricum M1. The Bacillus coagulans exosome could be obtained from Bacillus coagulans CC212 based on the aforementioned experiment and measurement, wherein a particle size and a concentration of the Bacillus coagulans exosome are basically identical to the particle size and the concentration of the Clostridium butyricum exosome.

4. Determining a concentration and an acid and alkali resistance of the Clostridium butyricum endospores and the Bacillus coagulans endospores:

Clostridium butyricum M1 and Bacillus coagulans CC212 are cultivated for sporulation:

Bacillus coagulans CC212 and Clostridium butyricum M1 are jointly inoculated in a bioreactor. A fermentation condition is as follows: Bacillus coagulans CC212 is inoculated at a ratio of 4%; Clostridium butyricum M1 is inoculated at a ratio of 6%; as both Bacillus coagulans CC212 and Clostridium butyricum M1 are anaerobic bacteria, the bioreactor is set to be in an anaerobic condition; a fermentation medium in the bioreactor has a temperature of 37° C. and a pH value maintained at 5.5. The bioreactor is left for cultivation for 48 hours; finally, a number of viable bacteria and a spore concentration of Clostridium butyricum M1 and Bacillus coagulans CC212 are analyzed by using a microscope.

Table 5 shows sporulation rates of Clostridium butyricum M1 and Bacillus coagulans CC212. The sporulation rate of Clostridium butyricum M1 is 86 wt %, indicating that the content of the Clostridium butyricum endospores accounts for 86 wt % of the combined content of Clostridium butyricum M1 and the Clostridium butyricum endospores, and the content of Clostridium butyricum M1 accounts for 14 wt % of the combined content of Clostridium butyricum M1 and the Clostridium butyricum endospores. The sporulation rate of Bacillus coagulans CC212 is 90 wt %, indicating that the content of the Bacillus coagulans endospores accounts for 90 wt % of the combined content of Bacillus coagulans CC212 and the Bacillus coagulans endospores, and the content of the Bacillus coagulans CC212 accounts for 10 wt % of the combined content of Bacillus coagulans CC212 and the Bacillus coagulans endospores.

TABLE 5
data table of the sporulation rates of Clostridium
butyricum M1 and Bacillus coagulans CC212

| | Total | | |
|---|---|---|---|
| | microbial | Number of | Sporulation |
| Item | number | spores | rate |
| Clostridium | 1.18 × 10⁹ | 1.02 × 10⁹ | 86 wt % |
| butyricum M1 | | | |
| Bacillus | 1.18 × 10⁹ | 1.62 × 10⁹ | 90 wt % |
| coagulans | | | |
| CC212 | | | |

Resistance experiment simulating the Clostridium butyricum endospores in digestive environments:

A gastrointestinal tract of an animal includes digestive environments of strong acidity in stomach, strong alkalinity in intestines, and bile salt, so that probiotics entering the digestive environments have difficulty in surviving and the viability of the strains is greatly reduced. The current experiment determines the resistance of the Clostridium butyricum endospores to acidic conditions, simulating the stomach of the animal, and the bile salt, simulating the small intestine of the animal.

### Acid Resistance Experiment:

0.5 g of the Clostridium butyricum endospores is dissolved in 20 ml of BHI medium; an anaerobic test tube of a control group and an anaerobic test tube of an experimental group are prepared; an additional 10 ml of BHI medium is added to the control group and the experimental group; a pH value of the BHI medium of the control group is regulated at 7.4; a pH value of the BHI medium of the experimental group is regulated at 2.0; afterwards, 1 ml of the BHI medium containing the Clostridium butyricum endospores is added to the BHI medium of the control group (pH=7.4) and the BHI medium of the experimental group (pH=2.0), respectively; then, the control group and the experimental group are placed in an incubator for cultivation at 37° C. for 3 hours; a bacterial solution of the control group is obtained from the anaerobic test tube of the control group and then is diluted; a bacterial solution of the experimental group is obtained from the anaerobic test tube of the experimental group and then is diluted; the diluted bacterial solution of the control group and the diluted bacterial solution of the experimental group are respectively spread on culture dishes; the culture dish of the control group and the culture dish of the experimental group are placed in an anaerobic chamber for cultivation for 16 hours to 24 hours; finally, numbers of colonies of the control group and the experimental group are calculated.

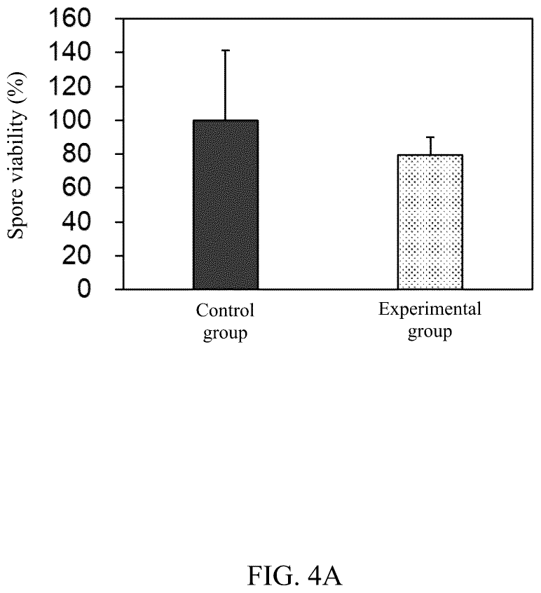

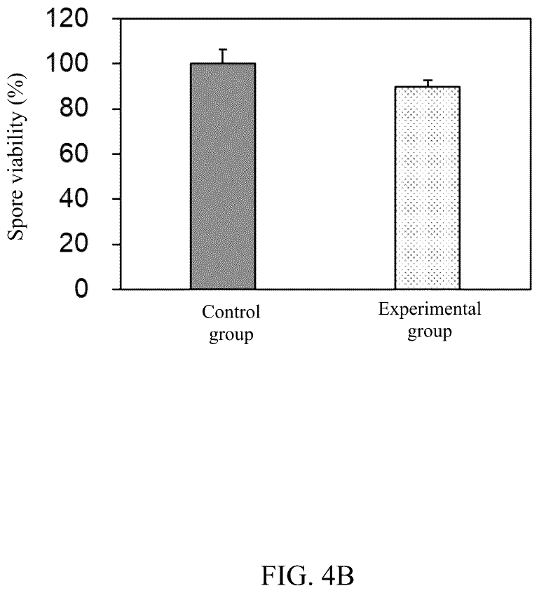

Measurement results are shown in FIG. 4A. The spore viability of the experimental group is 79% compared to the control group, indicating that the Clostridium butyricum endospores have a strong resistance to an environment at pH 2.0, i.e., the Clostridium butyricum endospores could survive in the highly acidic environment in the stomach of the animal.

### Bile Salt Resistance Experiment:

0.5 g of the Clostridium butyricum endospores are dissolved in 20 ml of BHI medium; an anaerobic test tube of a control group and an anaerobic test tube of an experimental group are prepared; an additional 10 ml of BHI medium is added to the control group and the experimental group; the BHI medium of the control group does not include bile salt (0% bile salt); the BHI medium of the experimental group includes 0.3% bile salt; afterwards, 1 ml of the BHI medium containing the Clostridium butyricum endospores is added to the BHI medium of the control group and the BHI medium of the experimental group, respectively; then the control group and the experimental group are placed in an incubator for cultivation at 37° C. for 4 hours; a bacterial solution of the control group is obtained from the anaerobic test tube of the control group and then is diluted; a bacterial solution of the experimental group is obtained from the anaerobic test tube of the experimental group and then is diluted; the diluted bacterial solution of the control group and the diluted bacterial solution of the experimental group are respectively spread on culture dishes; the culture dish of the control group and the culture dish of the experimental group are placed in an anaerobic chamber for cultivation for 16 hours to 24 hours; finally, numbers of colonies of the control group and the experimental group are calculated.

Measurement results are shown in FIG. 4B. The spore viability of the experimental group is 89% compared to the control group, indicating that the Clostridium butyricum endospores have a strong resistance to a bile salt environment, i.e., the Clostridium butyricum endospores could survive in the small intestine of the animal.

In summary, the Clostridium butyricum endospores have the strong resistance to acidic and alkaline environments, so that the Clostridium butyricum endospores could pass through the stomach and the small intestine of the animal. When the Clostridium butyricum endospores are situated in a condition favorable for growth, the Clostridium butyricum endospores undergo germination to reproduce Clostridium butyricum M1, thereby promoting the growth of Clostridium butyricum M1 in the intestines of the animal. Resistances of the Bacillus coagulans endospores to acidic and alkaline environments could be verified through the aforementioned experiments. The resistances of the Bacillus coagulans endospores to the acidic condition and the bile salt environment are basically identical to the resistances of the Clostridium butyricum endospores.

It must be pointed out that the embodiment described above is only a preferred embodiment of the present invention. All equivalent methods and structures which employ the concepts disclosed in this specification and the appended claims should fall within the scope of the present invention.

What is claimed is:

1. A composition for treating and/or relieving sarcopenia, comprising Clostridium butyricum M1.

2. The composition as claimed in claim 1, further comprising Clostridium butyricum endospores, wherein the Clostridium butyricum endospores are formed by Clostridium butyricum M1; a content of the Clostridium butyricum endospores accounts for between 30 wt % and 90 wt % of a total content of the composition; a content of Clostridium butyricum M1 accounts for between 10 wt % and 70 wt % of the total content of the composition.

3. The composition as claimed in claim 1, wherein the composition is added to a food of a subject; based on a weight of the food of the subject, an amount of the composition added ranges from 0.05 wt % to 1.0 wt %.

4. The composition as claimed in claim 2, wherein the composition is added to a food of a subject; based on a weight of the food of the subject, an amount of the composition added ranges from 0.05 wt % to 1.0 wt %.

5. The composition as claimed in claim 1, further comprising Bacillus coagulans CC212, wherein Bacillus coagulans CC212 is deposited in Bioresource Collection and Research Center; a deposit number of Bacillus coagulans CC212 is BCRC911233.

6. The composition as claimed in claim 5, wherein a weight ratio of Bacillus coagulans CC212 to Clostridium butyricum M1 is between 1:0.05 and 1:10.

7. The composition as claimed in claim 6, wherein a content of Clostridium butyricum M1 accounts for between 5 wt % and 95 wt % of a total content of the composition; a content of Bacillus coagulans CC212 accounts for between 5 wt % and 95 wt % of the total content of the composition; the composition is added to a food of a subject; based on a weight of the food of the subject, an amount of the composition added ranges from 0.05 wt % to 1.0 wt %.

8. The composition as claimed in claim 5, wherein the composition comprises Bacillus coagulans endospores and Clostridium butyricum endospores; the Bacillus coagulans endospores are formed by Bacillus coagulans CC212; the Clostridium butyricum endospores are formed by Clostridium butyricum M1; a ratio of a combined weight of Bacillus coagulans CC212 and the Bacillus coagulans endospores to a combined weight of Clostridium butyricum M1 and the Clostridium butyricum endospores is between 1:0.05 and 1:10.

9. The composition as claimed in claim 8, wherein a content of the Clostridium butyricum endospores accounts for between 30 wt % and 90 wt % of a combined content of Clostridium butyricum M1 and the Clostridium butyricum endospores; a content of Clostridium butyricum M1 accounts for between 10 wt % and 70 wt % of the combined content of Clostridium butyricum M1 and the Clostridium butyricum endospores; a content of the Bacillus coagulans endospores accounts for between 50 wt % and 90 wt % of a combined content of Bacillus coagulans CC212 and the Bacillus coagulans endospores; a content of Bacillus coagulans CC212 accounts for between 10 wt % and 50 wt % of the combined content of Bacillus coagulans CC212 and the Bacillus coagulans endospores; the composition is added to a food of a subject; based on a weight of the food of the subject, an amount of the composition added ranges from 0.05 wt % to 1.0 wt %.

10. A method of treating and/or relieving sarcopenia by using a composition for treating and/or relieving sarcopenia, the method comprising:
orally administering the composition to a subject, wherein the composition comprises Clostridium butyricum M1; Clostridium butyricum M1 releases a Clostridium butyricum exosome in the subject, the Clostridium butyricum exosome comprises butyric acid to activate vitamin B6 of the subject, thereby increasing a muscle mass of the subject.

11. The method as claimed in claim 10, wherein the composition comprises Clostridium butyricum endospores, the Clostridium butyricum endospores are formed by Clostridium butyricum M1; when the composition is administered to the subject, the Clostridium butyricum endospores undergo germination and release the Clostridium butyricum exosome; the composition is orally administered by adding the composition to a food of the subject for feeding; based on a weight of the food of the subject, an amount of the composition added ranges from 0.05 wt % to 1.0 wt %.

12. The method as claimed in claim 10, wherein the composition comprises Bacillus coagulans CC212; Bacillus coagulans CC212 is deposited in Bioresource Collection and Research Center; a deposit number of Bacillus coagulans CC212 is BCRC911233; when the composition is administered to the subject, Bacillus coagulans CC212 and Clostridium butyricum M1 are activated in the subject and Bacillus coagulans CC212 releases a Bacillus coagulans exosome in the subject; the Bacillus coagulans exosome and the Clostridium butyricum exosome are absorbed by the subject, thereby decreasing a fat mass of the subject.

13. The method as claimed in claim 12, wherein a weight ratio of Bacillus coagulans CC212 to Clostridium butyricum M1 is between 1:0.05 and 1:10.

14. The method as claimed in claim 13, wherein a content of Clostridium butyricum M1 accounts for between 5 wt % and 95 wt % of a total content of the composition; a content of Bacillus coagulans CC212 accounts for between 5 wt % and 95 wt % of the total content of the composition; the composition is orally administered by adding the composition to a food of the subject for feeding; based on a weight of the food of the subject, an amount of the added composition ranges from 0.05 wt % to 1.0 wt %.

15. The method as claimed in claim 12, wherein the composition comprises Bacillus coagulans endospores and Clostridium butyricum endospores; the Bacillus coagulans endospores are formed by Bacillus coagulans CC212; the Clostridium butyricum endospores are formed by Clostridium butyricum M1; when the composition is administered to the subject; the Bacillus coagulans endospores undergo germination and release the Bacillus coagulans exosome; the Clostridium butyricum endospores undergo germination and release the Clostridium butyricum exosome; a ratio of a combined weight of Bacillus coagulans CC212 and the Bacillus coagulans endospores to a combined weight of Clostridium butyricum M1 and the Clostridium butyricum endospores is between 1:0.05 and 1:10; the composition is orally administered by adding the composition to a food of the subject for feeding; based on a weight of the food of the subject, an amount of the composition added ranges from 0.05 wt % to 1.0 wt %.
